(19) United States
(12) Patent Application Publication
(10) Pub. No.: US 2004/0048381 A1
(43) Pub. Date: Mar. 11, 2004

(54) Vector

(76) Inventors: Johann Winkler, Macclesfield Cheshire (GB); Kenneth Robert Bundell, Macclesfield Cheshire (GB)

Correspondence Address: Janis K Fraser, Fish & Richardson, 225 Franklin Street, Boston, MA, 02110-2804, US

(21) Appl. No.: 10/416,128

(22) PCT Filed: Nov. 6, 2001
(86) PCT No.: PCT/GB01/04916

(30) Foreign Application Priority Data
Nov. 10, 2000 (GB) 0027501.6

(51) Int. Cl. C12N 15/86; C12N 15/74

(52) U.S. Cl. 435/456; 435/472

(57) ABSTRACT

The invention provides a two component system for in vitro cloning of a heterologous polynucleotide into adenoviral DNA. The first component is an insert donor comprising a heterologous polynucleotide encoding a heterologous polypeptide. The second component is a vector donor comprising an adenovirus genome and an expression cassette. The insert donor and vector donor are adapted for site specific recombination using recombination sites from phage lambda for insertion of the heterologous polynucleotide into the expression cassette capable of forming an adenoviral expression clone in vitro in the presence of a suitable recombination mediator protein or proteins. The invention also provides a method of making recombinant adenovirus and use of the first and second components in such a method.

Adenoviruses are a group of DNA viruses which can cause generally mild infections in humans respiratory illness, conjunctivitis and infantile gastroenteritis. Easily grown in cell culture, adenoviruses have been widely studied for many years (e.g. RNA splicing was first described in adenovirus infected cell). The ability of adenoviruses to infect human cells at up to 100% efficiency has led to its use as a vector for introducing foreign (recombinant) DNA in both cell culture and in humans (gene therapy). Recombinant adenoviruses generally have certain regions of DNA deleted (e.g. E1 region necessary for replication, E3 required for evading host immunity). The purpose of this is twofold. First, the removal of non-necessary regions of DNA allows space for the introduction of foreign DNA which can then be packaged as adenovirus DNA and subsequently introduced into human cells (there is also some leeway for insertion of extra DNA into the genome without affecting infectivity). Second, the removal of the E1 region determines that the recombinant virus can infect human cells but not replicate; this is an important safety consideration. To generate and replicate recombinant adenovirus, DNA is transfected into a cell line (e.g. HEK 293) which has the E1 region engineered into its genome i.e. the cell line provides the replicative machinery lacking in the recombinant adenovirus.

Recombinant adenovirus technology is often based around human adenovirus type 5. The genome for Ad5 is 35.935 kb. This whole sequence can be cloned into a plasmid vector and replicated in E. coli. However, because of the length of adenovirus sequence, there are very few suitable restriction enzyme sites available which would allow direct cloning into such a vector. To circumvent this problem, a two vector approach has been adopted. One vector contains the complete adenovirus sequence minus E1 and E3 sequence. The second vector contains a eukaryotic promoter (e.g. CMV) upstream of a cloning site (for insertion of foreign DNA), and polyadenylation sequences necessary for stability of transcribed RNA. Flanking this expression cassette are two regions of adenovirus DNA which are common with adenovirus sequences in the first vector. Thus, the expression cassette from the second vector can be introduced into the adenovirus sequence in the first vector by a process of homologous recombination. This tedious, exacting and time-consuming process has traditionally been performed in HEK 293 cells. Viruses produced have to be plaque purified, propagated and tested to ensure that the desired recombinant DNA has been introduced.

A recent advance in recombinant adenovirus generation, the ‘AdEasy™’ system (He, T-G et al, PNAS, 95, 1998, 2509-2514), simplifies the procedure by carrying out the plasmid recombination step in E. coli strain BJ 5183 which allows limited recombination.

Recombinant adenoviral DNA can then be identified by restriction digest and clonal adenoviral DNA prepared in E. coli. The clonal adenoviral DNA can then be transfected into HEK 293 cells and adenovirus produced thus effectively removing the need for plaque purification. This method has now been commercialised (Q-biogene: Adeasy™. A further improvement with this system is that the recombinant adenovirus co-expresses green fluorescent protein from a second CMV promoter. This makes infection efficiency (and therefore gene delivery efficiency) simple to assess. Our experience with the Adeasy™ system has shown that there can be difficulties with the recombination procedure. This can involve high background from the expression vector, unpredictable recombination events and low recombination efficiency. Thus there is a need for further improvements in recombinant adenovirus generation.

According to one aspect of the present invention there is provided a two component system for in vitro cloning of a heterologous polynucleotide into adenoviral DNA comprising:

i) a first component which is an insert donor comprising a heterologous polynucleotide encoding a heterologous polypeptide; and

ii) a second component which is a vector donor comprising an adenovirus genome and an expression cassette;

wherein the insert donor and vector donor are adapted for site specific recombination for insertion of the heterologous polynucleotide into the expression cassette capable of forming an adenoviral expression clone in vitro in the presence of a suitable recombination mediator protein or proteins.

Preferably the site specific recombination uses recombination sites from phage lambda. For a review of recombination in lambda the reader is referred to the following: Landy (1989) Ann Rev Biochem 58, 913; and Ptashne (1992) A Genetic Switch, Cell Press, Cambridge. The reader is also referrred to U.S. Pat. No. 5,888,732 (Life Technologies) for further technical details of site specific recombination using insert donor and vector donor moieties. In use, the recombination reactions produce highly specific cutting and ligation reactions such that the recombination mediator proteins cut to the left and right of the heterologous polynucleotide in the insert donor and ligate it into the vector donor whereby to form an adenoviral expression clone.

Preferably the expression cassette comprises a polynucleotide encoding a fluorescent protein downstream of an internal ribosome entry site for expression from the same mRNA as the heterologous polypeptide.

Preferably system described herein comprises at least one of the following:

i) a CMV promoter in the expression cassette;

ii) an adenovirus type 5 genome that is replication deficient;

iii) a vector donor comprising a ccdB gene;

iv) an insert donor comprising a selectable marker;

v) a vector donor comprising a selectable marker; and

vi) site specific recombination sites based on either phage lambda attB with attP or attL with attR.

More preferably the system comprises all of the elements i) to vi). The attB x attP reaction is mediated by proteins Int and IHF (Clonase BP™, Life Technologies). The attL x attR reaction is mediated by proteins Int, IHF, and Xis (Clonase LR™, Life Technologies). Int and Xis are from lambda; IHF is from E. coli. Here “x” represents recombination. Engineered recombination sites offereing efficiency or specificity advantages over wild type sequences as described in U.S. Pat. No. 5,888,732 are also contemplated.

According to another aspect of the invention there is provided a method of making recombinant adenovirus comprising:

i) mixing in vitro a first component as defined herein with a second component as defined herein in the presence of a suitable recombination mediator protein or proteins so as to form an expression clone;

ii) transformation of product from i) in to a host organism suitable for expression clone replication; and

iii) transfection of product from ii) in to a host suitable for adenoviral replication.

Preferably the method uses first and second components as defined in elements i) to vi) above, the host organism for expression clone replication is E. coli and the host organism for adenoviral replication is HEK 293 cells.

According to another aspect of the invention there is provided a second component as defined herein.

According to another aspect of the invention there is provided use of a first component as defined herein in a method as defined herein.

According to another aspect of the invention there is provided the use of a second component as defined herein in a method as defined herein.

The invention is illustrated below by the following non-limiting Example in which:

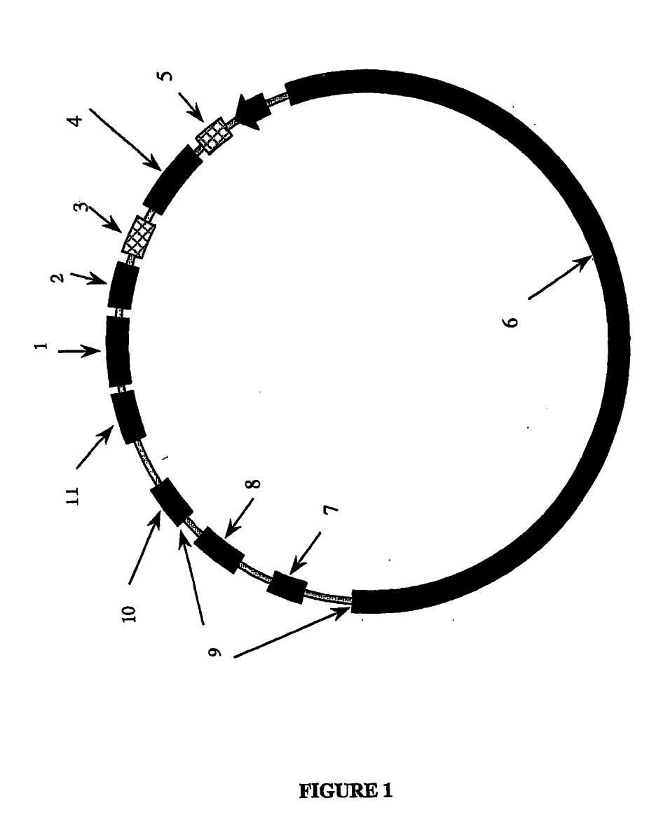

FIG. 1 shows a Vector Donor wherein 1=fluorescent protein, 2=IRES element, 3=attR2, 4=ccdB+cm^{R}, 5=attR1, 6=adenovirus sequence (ΔE1/ΔE3), 7=origin of replication, 8=ampicillin resistance, 9=restriction enzyme sites for excision of plasmid backbone, 10=adenovirus LITR sequence, and 11=SV40 PA;

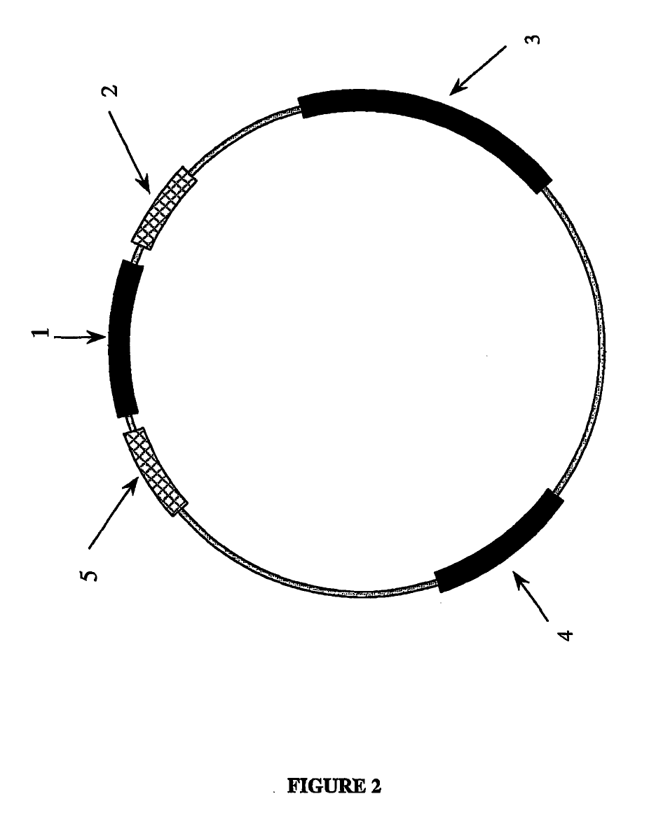

FIG. 2 shows an Insert Donor in which 1=gene of interest, 2=attL2, 3=kanamycin resistance, 4=origin of replication, 5=attL1.

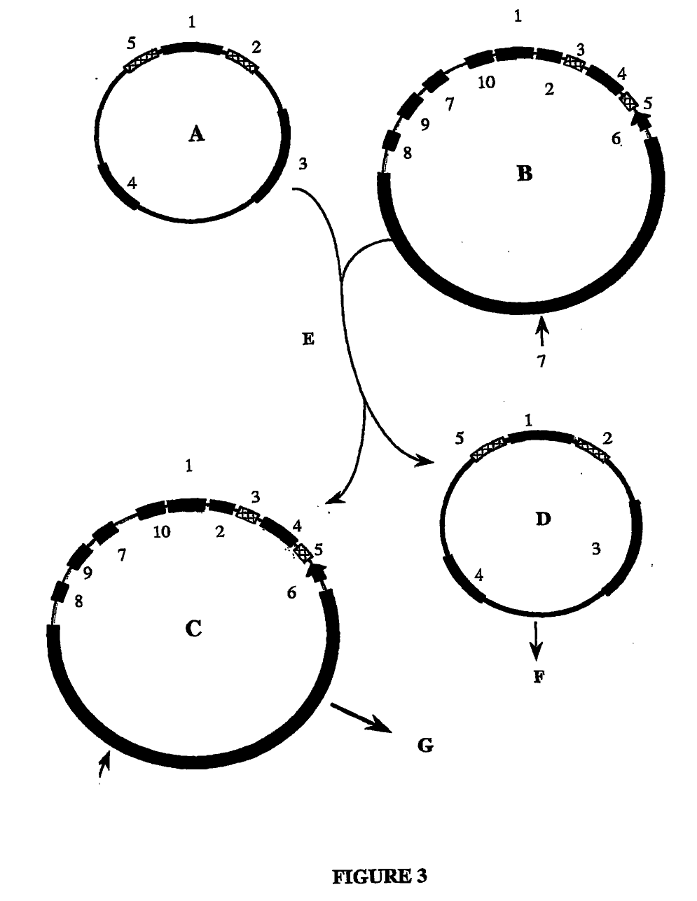

FIG. 3 shows in vitro cloning of lac Z into an adenoviral expression clone wherein A=Insert Donor wherein 1=gene of interest (lacZ), 2=attL2, 3=Kn^{r}, 4=ori, 5=attL1. B=Vector Donor wherein 1=fluorescent protein, 2=IRES, 3=attR2, 4=ccdB, 5=attR1, 6=CMV promoter, 7=adenovirus sequence, 8=ori, 9=Amp^{r}, 10=SV40 PA. C=Expression clone (Amp^{r}) wherein 1=fluorescent protein, 2=IRES, 3=AttB2, 4=lacZ, 5=AttB1, 6=CMV promoter, 7=adenovirus sequence, 8=ori, 9=Amp², 10=SV40 PA. D=Bi-product (kana^{r}) wherein 1=ccdB, 2=AttP2, 3=Kn^{r}, 4=ori, 5=AttP1. E=Int, IHF, Xis proteins. F=Will not grow on ampicillin or in E. coli DH5α (ccdB lethal). G=Transform E. coli (eg. DH5α) and select on ampicillin plates identify correct clones and transfect HEK 293 cells to generate virus.

General molecular biology techniques are described in “Current Protocols in Molecular Biology Volumes 1-3, edited by F M Asubel, R Brent and R E Kingston; published by John Wiley, 1998.

### EXAMPLE 1

We have used an in vitro cloning system to make recombinant adenovirus generation simpler and more efficient. Also we have incorporated fluorescent protein as infection marker (Matz et al (1999) Nat. Biotechnol. 17, 969-973; Lukyanov et al (2000) J. Biol. Chem. 275, 25879-25882).

In brief, we have created a vector which contains: the complete adenovirus genome (minus E1 and E3); an expression cassette comprising: CMV promoter, attR1-Chloramphenicol resistance−ccdB−attR2, internal ribosome entry site (IRES), fluorescent protein sequence, and SV40 polyadenylation sequence.

This vector is a vector donor. Any sequence in an insert donor can be efficiently cloned directly into this vector donor via in vitro recombination. Background is reduced to zero due to the ccdB gene toxicity in E. coli (the vector donor DNA is propagated in E. coli strain DB3.1 which has a gyrA mutation and tolerates ccdB). Thus in vitro recombination of the gene of interest in an insert donor into the vector donor followed by transformation in E. coli DH5a using selection for the vector donor (vector donor ampicillin resistant, insert donor kanamycin resistant) should only result in recombinant adenoviral DNA since the ccdB gene is toxic. The DNA is then digested to remove the plasmid backbone and directly transfected into HEK 293 cells to generate recombinant virus.

We have used this system to generate recombinant expressing lacZ from an insert donor containing lacZ. It is quick and efficient and we believe has a considerable advantage over the current AdEasy system. Also, these constructs express a fluorescent protein from an IRES element. This means that they are expressed from the same transcribed messenger RNA as the recombinant gene. With ‘AdEasy™’, the green fluorescent protein is expressed from a second separate CMV promoter. In this case expression of the fluorescent protein is no guarantee of recombinant gene transcription.

A 3168 base pair fragment comprising the entire coding region of the E. coli lacZ gene preceded by a sequence encoding six histidine residues was isolated from pZeoSV2/lacZ (Invitrogen) by restriction enzyme digest (Nco I and EcoR I). The lacZ DNA fragment was separated from the plasmid backbone by gel electrophoresis, excised from the gel and purified using Geneclean™ Spin Kit. The isolated fragment was then cloned into the Nco I and EcoR I sites of insert donor pENTR 11 (Life Technologies). Briefly, pENTR 11 was digested with Nco I and EcoR I, purified by gel excision and dephosphorylated with shrimp alkaline phosphatase (Roche). The lac Z fragment was then ligated to the pENTR 11 plasmid in vitro using T4 DNA ligase (Roche) and transformed into E. coli DH10B electrocompetent cells (Life Technologies). Following overnight growth on kanamycin plates, a pENTR 11 clone containing lacZ was identified by restriction digest of plasmid DNA. Thus this clone contained the lacZ coding sequence flanked by 1 bacteriophage attL1 and attL2 sites. For generation of adenoviral DNA containing the lacZ gene, vector donor comprising the following was prepared:

(i) Adenovirus DNA sequence (minus E1 and E3 regions)

(ii) Exciseable plasmid backbone (ampicillin resistant)

(iii) CMV promoter for expression of heterologous genes in mammalian cells

(iv) chloramphenicol resistance gene and E. coli ccdB gene flanked by 1 bacteriophage attR1 and attR2 sites

(v) an internal ribosome entry site (IRES) element

(vi) fluorescent protein coding sequences

(vii) SV40 polyadenylation sequence for mRNA stability.

This vector donor was incubated together with insert donor (pENTR 11/lacZ) in a buffer containing recombination mediator proteins Int, IHF, and Xis (LR clonase™, Life Technologies). The resultant DNA was transformed into E. coli DH10B electrocompetent cells and plated out on ampicillin plates. 15 colonies were selected from the several thousand present and analysed by restriction digest of prepared plasmid DNA. Of these, 14 (93%) contained the desired adenoviral lacZ construct and only 1 was not the desired construct.

For generation of adenoviruses expressing lacZ and fluorescent protein, adenoviral lacZ plasmid DNA was digested with Pac I to remove the plasmid backbone and transfected into HEK 293 cells using Lipofectamine™ (Life Technologies). After 12 days incubation, viral growth was apparent and virus was harvested from cells lysates.

### COMPARATIVE EXAMPLE 1

Recombinant adenoviral DNA generation using “AdEasy™” homologous recombination in E. coli BJ 5183 was much less productive and efficient than the in vitro method of Example 1. In general, far fewer colonies were obtained (<20 per transfection compared with many thousands) and of these fewer were the desired recombinant (typically 10-30%).

1 A two component system for in vitro cloning of a heterologous polynucleotide into adenoviral DNA comprising:
i) a first component which is an insert donor comprising a heterologous polynucleotide encoding a heterologous polypeptide; and
ii) a second component which is a vector donor comprising an adenovirus genome and an expression cassette;
wherein the insert donor and vector donor are adapted for site specific recombination for insertion of the heterologous polynucleotide into the expression cassette capable of forming an adenoviral expression clone in vitro in the presence of a suitable recombination mediator protein or proteins.

2 A system according to claim 1 wherein the site specific recombination uses recombination sites from phage lambda.

3. A system according to any previous claim in which the expression cassette comprises a polynucleotide encoding a fluorescent protein downstream of an internal ribosome entry site for expression from the same mRNA as the heterologous polypeptide.

4. A system according to any previous claim comprising at least one of the following:
i) a CMV promoter in the expression cassette;
ii) an adenovirus type 5 genome that is replication deficient;
iii) a vector donor comprising a ccdB gene;
iv) an insert donor comprising a selectable marker;
v) a vector donor comprising a selectable marker; and
vi) site specific recombination sites based on either phage lambda attB with attP or attL with attR.

5. A system according to claim 4 comprising all of the claimed elements i) to vi).

6. A method of making recombinant adenovirus comprising:
i) mixing in vitro a first component as defined in any one of claims 1-5 with a second component as defined in any one of claims 1-5 in the presence of a suitable recombination mediator protein or proteins so as to form an expression clone;
ii) transformation of product from i) in to a host organism suitable for expression clone replication; and
iii) transfection of product from ii) in to a host suitable for adenoviral replication.

7 A method according to claim 6 in which the first and second components are as defined in claim 5, the host organism for expression clone replication is E. coli and the host organism for adenoviral replication is HEK 293 cells.

8 A second component as defined in any one of claims 1-5.

9. Use of a first component as defined in any one of claims 1-5 in a method as defined in claim 6 or 7.

10. Use of a second component as defined in any one of claims 1-5 in a method as defined in claim 6 or 7.
